(19) United States
(12) Patent Application Publication
(10) Pub. No.: US 2023/0343435 A1
(43) Pub. Date: Oct. 26, 2023

(54) SYSTEMS AND METHODS FOR REQUESTING CONSENT FOR DATA

(71) Applicant: ResMed Sensor Technologies Limited, Dublin (IE)

(72) Inventors: Michael Wren, Dublin (IE); Redmond Shouldice, Dublin (IE)

(21) Appl. No.: 17/796,429

(22) PCT Filed: Jan. 28, 2021
(86) PCT No.: PCT/IB2021/050646

§ 371 Date: Jul. 29, 2022

Related U.S. Application Data
(60) Provisional application No. 62/968,777, filed on Jan. 31, 2020.

(51) Int. Cl. G16H 20/40; G16H 10/60; G16H 50/30; G16H 50/70; A61B 5/00; A61B 5/08; A61B 5/11

(52) U.S. Cl. CPC G16H 20/40; G16H 10/60; G16H 50/30; G16H 50/70; A61B 5/4833; A61B 5/4815; A61B 5/0816; A61B 5/0823; A61B 5/11

(57) ABSTRACT

A method of analyzing data related to use of a respiratory therapy system by a user comprises receiving a first type of data, determining a first value of a first parameter based at least in part on the first type of data, identifying a desired second type of data, transmitting a request for consent to receive the second type of data, and determining, based at least in part on the second type of data, a second value of the first parameter, a value of a second parameter, or both. The first type of data and the first parameter are related to the user's use of the respiratory therapy system. The identification of the second type of data is based at least in part on the first type of data, the first value of the first parameter, an accuracy of the first value of the first parameter, or any combination thereof.

### CROSS-REFERENCE TO RELATED APPLICATIONS

This application claims the benefit of, and priority to, U.S. Provisional Patent Application No. 62/968,777 filed on Jan. 31, 2020, which is hereby incorporated by reference herein in its entirety.

### TECHNICAL FIELD

The present disclosure relates generally to systems and methods for analyzing data related to a user using a respiratory therapy system, and more particularly, to systems and methods for obtaining consent to receive and analyze data related to the user using the respiratory therapy system.

### BACKGROUND

Many individuals suffer from sleep-related and/or respiratory-related disorders, such as insomnia (e.g., difficulty initiating sleep, frequent or prolonged awakenings after initially falling asleep, and an early awakening with an inability to return to sleep), periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), etc. Many of these disorders can be treated or managed more effectively if certain data about the individual is received and analyzed. Thus, it would be advantageous to efficiently obtain consent to receive and analyze data related to the individual. The present disclosure is directed to solving these and other problems.

### SUMMARY

According to some implementations of the present disclosure, a method of analyzing data related to a use of a respiratory therapy system by a user during a sleep session comprises receiving a first type of data related to the use of the respiratory therapy system by the user during the sleep session; determining a first value of a first parameter related to the use of the respiratory therapy system of the user based at least in part on the first type of data; identifying a desired second type of data; transmitting to the user a request for consent to receive the second type of data; in response to receiving consent from the user, receiving the second type of data; and determining, based at least in part on the second type of data, (i) a second value of the first parameter, (ii) a value of a second parameter, or (iii) both (i) and (ii).

According to some implementations of the present disclosure, a method of analyzing data related to a use of a respiratory therapy system by a user during a sleep session comprises receiving (i) a first type of data related to the user during the sleep session, and (ii) consent to analyze the first type of data to determine a value of a first parameter related to the user; determining the value of a first parameter related to the user based at least on the first type of data; identifying a desired second parameter; transmitting to the user a request for consent to analyze the first type of data to determine a value of the second parameter related to the user; and in response to receiving consent from the user, determining the value of the second parameter related to the user based at least on the first type of data.

According to some implementations of the present disclosure, a method of analyzing data associated with use of a plurality of respiratory therapy systems by a plurality of users comprises transmitting, to each respective user of the plurality of users, a plurality of requests for consent to receive data associated with a use of a respective one of the plurality of respiratory therapy systems by the respective user, the plurality of requests being transmitted to each respective user according to a respective order; in response to receiving consent, receiving data from two or more of the plurality of users; and analyzing the data received from each respective user of the two or more of the plurality of users to determine an optimal order for transmitting the plurality of requests for consent to receive the data.

According to some implementations of the present disclosure, a method of analyzing data related to use of a respiratory therapy system by a user during a current sleep session comprises storing a plurality of historical values of a first parameter related to the user; receiving a first type of data related to the user during the current sleep session; determining a current value of the first parameter based at least in part on the first type of data; comparing the current value of the first parameter and the plurality of historical values of the first parameter; in response to the comparison between the current value of the first parameter and the plurality of historical values of the first parameter satisfying a threshold, identifying a desired second type of data; and transmitting to the user a request for consent to receive the second type of data.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system for analyzing data related to a user using a respiratory therapy system, according to some implementations of the present disclosure;

FIG. 2 is a perspective view of the system of FIG. 1, a user of the system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 is a process flow diagram for a first method of analyzing data related to use of a respiratory therapy system, according to some implementations of the present disclosure;

FIG. 6 is a process flow diagram for a second method of analyzing data related to use of a respiratory therapy system, according to some implementations of the present disclosure;

FIG. 7 is a process flow diagram for a method of determining an optimal order for transmitting a plurality of requests for consent to receive and analyze data, according to some implementations of the present disclosure; and

FIG. 8 is a process flow diagram for a method of analyzing data related to use of a respiratory therapy system to determine a change in a parameter related to the user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Central Sleep Apnea (CSA), other types of apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. Central Sleep Apnea (CSA) is another form of SDB that results when the brain temporarily stops sending signals to the muscles that control breathing. More generally, an apnea generally refers to the cessation of breathing caused by blockage of the air or the stopping of the breathing function. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of SDB. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

A wide variety of types of data can be used to monitor the health of individuals having any of the above types of sleep-related and/or respiratory disorders (or other disorders). However, these individuals generally do not initially or automatically consent to providing the large amount of data that can be actually be utilized to monitor the individual's health. Instead, these individuals often consent initially to only providing limited data related to the individual's respiration while they are sleeping. Thus, it is advantageous to explain to the user why additional data is needed and how the additional data can be utilized, in order to receive proper informed consent to obtain and analyze the additional data.

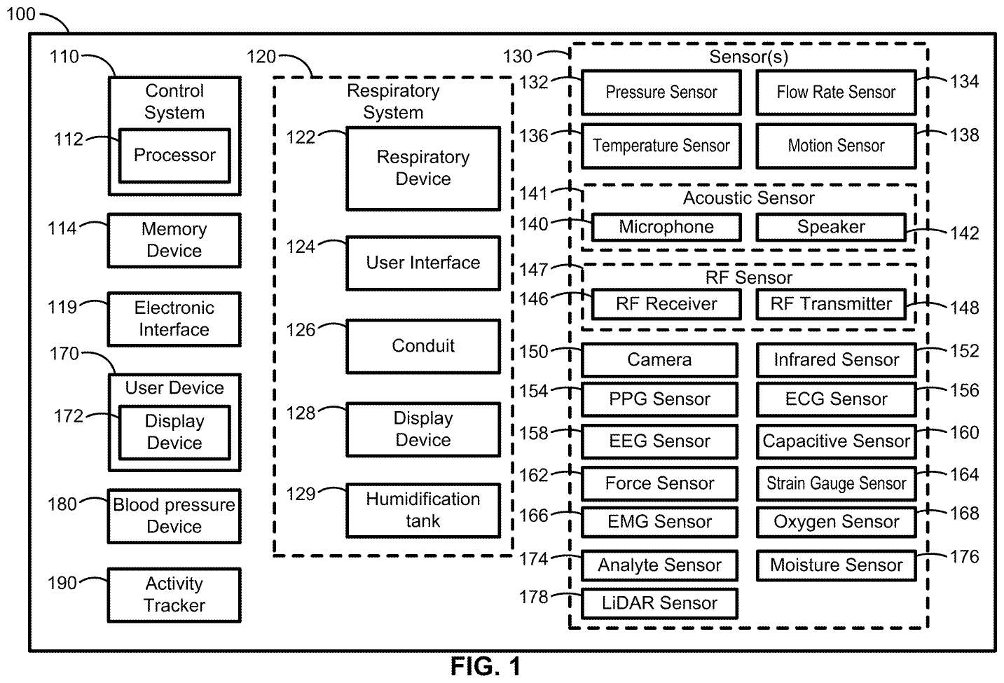

Referring to FIG. 1, a system 100, according to some implementations of the present disclosure, is illustrated. The system 100 is for providing a variety of different sensors related to a user's use of a respiratory therapy system, among other uses. The system 100 includes a control system 110, a memory device 114, an electronic interface 119, one or more sensors 130, and one or more external devices 170. In some implementations, the system 100 further includes a respiratory therapy system 120 (that includes a respiratory therapy device 122), a blood pressure device 180, an activity tracker 190, or any combination thereof. The system 100 can be used to analyze a variety of different types of data related to the user's use of the respiratory therapy system 120.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the external device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of any one or more of the sensors 130. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family medical history, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the external device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory pressure therapy system). The respiratory therapy system 120 can include a respiratory therapy device 122 (also referred to as a respiratory pressure therapy device), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea), other respiratory disorders such as COPD, or other disorders leading to respiratory insufficiency, that may manifest either during sleep or wakefulness.

The respiratory therapy device 122 is generally used to generate pressurized air that is delivered to a user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure). In some implementations, the control system 110, the memory device 114, the electronic interface 119, or any combination thereof can be coupled to and/or positioned within a housing of the respiratory therapy device 122.

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

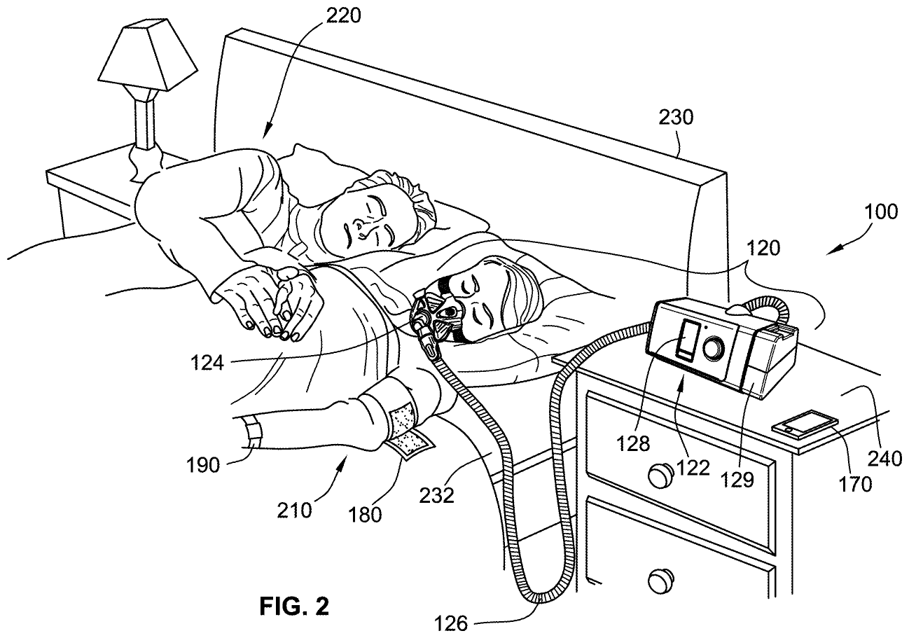

In some implementations, the user interface 124 is or includes a facial mask that covers the nose and mouth of the user (as shown, for example, in FIG. 2). Alternatively, the user interface 124 is or includes a nasal mask that provides air to the nose of the user or a nasal pillow mask that delivers air directly to the nostrils of the user. The user interface 124 can include a strap assembly that has a plurality of straps (e.g., including hook and loop fasteners) for positioning and/or stabilizing the user interface 124 on a portion of the user interface 124 on a desired location of the user (e.g., the face), and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user. In other implementations, the user interface 124 includes a mouthpiece (e.g., a night guard mouthpiece molded to conform to the user's teeth, a mandibular repositioning device, etc.).

The conduit 126 allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (also referred to as a myAir™ score, such as described in WO 2016/061629, which is hereby incorporated by reference herein in its entirety), the current date/time, personal information for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. In other implementations, the respiratory therapy device 122 or the conduit 126 can include a waterless humidifier. The waterless humidifier can incorporate sensors that interface with other sensor positioned elsewhere in system 100.

The respiratory therapy system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based at least in part on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, an RF transmitter 148, a camera 150, an infrared (IR) sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a light detection and ranging (LiDAR) sensor 178, or any combination thereof. Generally, each of the one or sensors 130 are configured to output sensor data that is received and stored in the memory device 114 or one or more other memory devices. The sensors 130 can also include, an electrooculography (EOG) sensor, a peripheral oxygen saturation (SpO₂) sensor, a galvanic skin response (GSR) sensor, a carbon dioxide (CO₂) sensor, or any combination thereof.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example physiological data, acoustic data, or both, that is associated with a user of the respiratory therapy system 120 (such as the user 210 of FIG. 2), the respiratory therapy system 120, both the user and the respiratory therapy system 120, or other entities, objects, activities, etc. Physiological data generated by one or more of the sensors 130 can be used by the control system 110 to determine a sleep-wake signal associated with the user during the sleep session and one or more sleep-related parameters. The sleep-wake signal can be indicative of one or more sleep stages and/or sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep stages and/or sleep states from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The sleep-wake signal can also be timestamped to indicate a time that the user enters the bed, a time that the user exits the bed, a time that the user attempts to fall asleep, etc. The sleep-wake signal can be measured one or more of the sensors 130 during the sleep session at a predetermined sampling rate, such as, for example, one sample per second, one sample per 30 seconds, one sample per minute, etc. Examples of the one or more sleep-related parameters that can be determined for the user during the sleep session based at least in part on the sleep-wake signal include a total time in bed, a total sleep time, a total wake time, a sleep onset latency, a wake-after-sleep-onset parameter, a sleep efficiency, a fragmentation index, an amount of time to fall asleep, a consistency of breathing rate, a fall asleep time, a wake time, a rate of sleep disturbances, a number of movements, or any combination thereof.

Physiological data and/or acoustic data generated by the one or more sensors 130 can also be used to determine a respiration signal associated with the user during a sleep session. the respiration signal is generally indicative of respiration or breathing of the user during the sleep session. The respiration signal can be indicative of, for example, a respiration rate, a respiration rate variability, an inspiration amplitude, an expiration amplitude, an inspiration-expiration amplitude ratio, an inspiration-expiration duration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, an elevated stress level, etc.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of the user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user, a skin temperature of the user, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume.

The microphone 140 outputs acoustic data that can be stored in the memory device 114 and/or analyzed by the processor 112 of the control system 110. The acoustic data generated by the microphone 140 is reproducible as one or more sound(s) during a sleep session (e.g., sounds from the user) to determine (e.g., using the control system 110) one or more sleep-related parameters, as described in further detail herein. The acoustic data from the microphone 140 can also be used to identify (e.g., using the control system 110) an event experienced by the user during the sleep session, as described in further detail herein. In other implementations, the acoustic data from the microphone 140 is representative of noise associated with the respiratory therapy system 120. The microphone 140 can be coupled to or integrated in the respiratory therapy system 120 (or the system 100) generally in any configuration. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the external device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway.

The speaker 142 outputs sound waves that are audible to the user. The speaker 142 can be used, for example, as an alarm clock or to play an alert or message to the user (e.g., in response to an event). In some implementations, the speaker 142 can be used to communicate the acoustic data generated by the microphone 140 to the user. The speaker 142 can be coupled to or integrated in the respiratory therapy device 122, the user interface 124, the conduit 126, or the external device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user or a bed partner of the user (such as bed partner 220 in FIG. 2). Based at least in part on the data from the microphone 140 and/or the speaker 142, the control system 110 can determine a location of the user and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the external device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based at least in part on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user enters the user's bed (such as bed 230 in FIG. 2), and to determine a time when the user exits the bed 230. The camera 150 can also be used to track eye movements, pupil dilation (if one or both of the user's eyes are open), blink rate, or any changes during REM sleep. The camera 150 can also be used to track the position of the user, which can impact the duration and/or severity of apneic episodes in users with positional obstructive sleep apnea.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user, embedded in clothing and/or fabric that is worn by the user, embedded in and/or coupled to the user interface 124 and/or its associated headgear (e.g., straps, etc.), etc.

The ECG sensor 156 outputs physiological data associated with electrical activity of the heart of the user. In some implementations, the ECG sensor 156 includes one or more electrodes that are positioned on or around a portion of the user during the sleep session. The physiological data from the ECG sensor 156 can be used, for example, to determine one or more of the sleep-related parameters described herein.

The EEG sensor 158 outputs physiological data associated with electrical activity of the brain of the user. In some implementations, the EEG sensor 158 includes one or more electrodes that are positioned on or around the scalp of the user during the sleep session. The physiological data from the EEG sensor 158 can be used, for example, to determine a sleep stage and/or a sleep state of the user at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user. The data output by the analyte sensor 174 can be stored in the memory device 114 and used by the control system 110 to determine the identity and concentration of any analytes in the user's breath. In some implementations, the analyte sensor 174 is positioned near a mouth of the user to detect analytes in breath exhaled from the user's mouth. For example, when the user interface 124 is a facial mask that covers the nose and mouth of the user, the analyte sensor 174 can be positioned within the facial mask to monitor the user mouth breathing. In other implementations, such as when the user interface 124 is a nasal mask or a nasal pillow mask, the analyte sensor 174 can be positioned near the nose of the user to detect analytes in breath exhaled through the user's nose. In still other implementations, the analyte sensor 174 can be positioned near the user's mouth when the user interface 124 is a nasal mask or a nasal pillow mask. In this implementation, the analyte sensor 174 can be used to detect whether any air is inadvertently leaking from the user's mouth. In some implementations, the analyte sensor 174 is a volatile organic compound (VOC) sensor that can be used to detect carbon-based chemicals or compounds, such as carbon dioxide. In some implementations, the analyte sensor 174 can also be used to detect whether the user is breathing through their nose or mouth. For example, if the data output by an analyte sensor 174 positioned near the mouth of the user or within the facial mask (in implementations where the user interface 124 is a facial mask) detects the presence of an analyte, the control system 110 can use this data as an indication that the user is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user's face, near the connection between the conduit 126 and the user interface 124, near the connection between the conduit 126 and the respiratory therapy device 122, etc.). Thus, in some implementations, the moisture sensor 176 can be coupled to or integrated into the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user, for example the air inside the user's bedroom. The moisture sensor 176 can also be used to track the user's biometric response to environmental changes.

One or more LiDAR sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the external device 170, or any combination thereof. For example, the acoustic sensor 141 and/or the RF sensor 147 can be integrated in and/or coupled to the external device 170. In such implementations, the external device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory therapy device 122. In some implementations, at least one of the one or more sensors 130 is not coupled to the respiratory therapy device 122, the control system 110, or the external device 170, and is positioned generally adjacent to the user during the sleep session (e.g., positioned on or in contact with a portion of the user, worn by the user, coupled to or positioned on the nightstand, coupled to the mattress, coupled to the ceiling, etc.). More generally, the one or more sensors 130 can be positioned at any suitable location relative to the user such that the one or more sensors 130 can generate physiological data associated with the user and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The external device 170 includes a display device 172. The external device 170 can be, for example, a mobile device such as a smart phone, a tablet, a laptop, or the like. Alternatively, the external device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the external device 170. In some implementations, one or more user devices can be used by and/or included in the system 100.

The blood pressure device 180 is generally used to aid in generating physiological data for determining one or more blood pressure measurements associated with a user. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by a user and a pressure sensor (e.g., the pressure sensor 132 described herein). For example, as shown in the example of FIG. 2, the blood pressure device 180 can be worn on an upper arm of the user. In such implementations where the blood pressure device 180 is a sphygmomanometer, the blood pressure device 180 also includes a pump (e.g., a manually operated bulb) for inflating the cuff. In some implementations, the blood pressure device 180 is coupled to the respiratory therapy device 122 of the respiratory therapy system 120, which in turn delivers pressurized air to inflate the cuff. More generally, the blood pressure device 180 can be communicatively coupled with, and/or physically integrated in (e.g., within a housing), the control system 110, the memory device 114, the respiratory therapy system 120, the external device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user. The activity tracker 190 can also be coupled to or integrated a garment or clothing that is worn by the user. Alternatively, still, the activity tracker 190 can also be coupled to or integrated in (e.g., within the same housing) the external device 170. More generally, the activity tracker 190 can be communicatively coupled with, or physically integrated in (e.g., within a housing), the control system 110, the memory device 114, the respiratory therapy system 120, the external device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the external device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for canceling noises during use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the external device 170. As yet another example, a third alternative system includes the control system 110, the memory device 114, the respiratory therapy system 120, at least one of the one or more sensors 130, and the external device 170. As a further example, a fourth alternative system includes the control system 110, the memory device 114, the respiratory therapy system 120, at least one of the one or more sensors 130, the external device 170, and the blood pressure device 180 and/or activity tracker 190. Thus, various systems for analyzing data related to the user's use of the respiratory therapy system 120 can be formed using any portion or portions of the components shown and described herein and/or in combination with one or more other components.

As used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the external device 170 (FIG. 1) to manually initiate or terminate the sleep session.

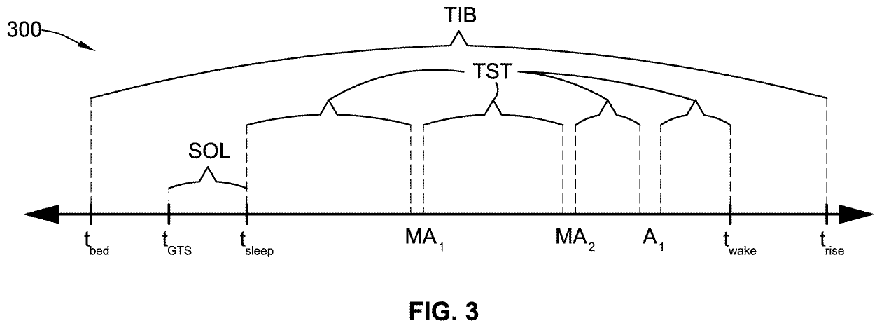

Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the external device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

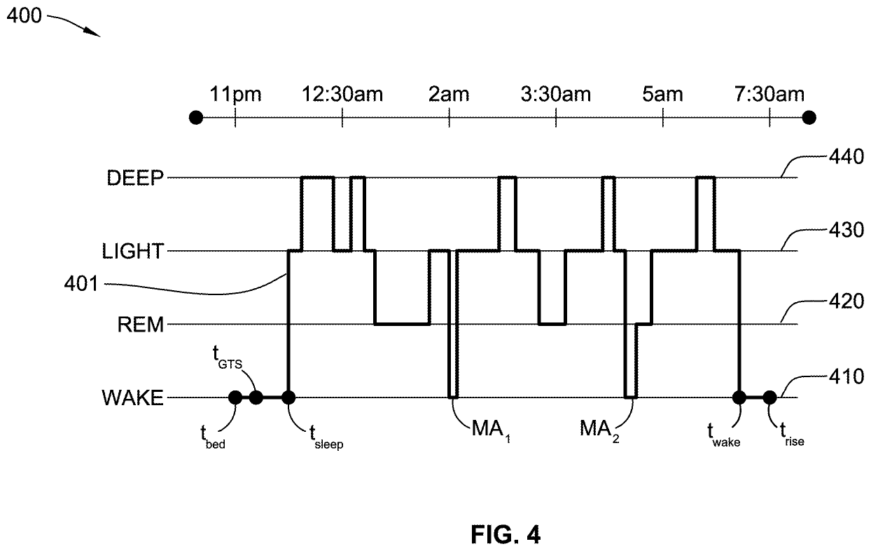

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 130 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 138, the microphone 140, the camera 150, or any combination thereof. The go-to-sleep time can be determined based at least in part on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the external device 170 (e.g., data indicative of the user no longer using the external device 170), data from the pressure sensor 132 and/or the flow rate sensor 134 (e.g., data indicative of the user turning on the respiratory therapy device 122, data indicative of the user donning the user interface 124, etc.), or any combination thereof.

When the user uses the respiratory therapy system 120 during a sleep session, a large amount of data can be generated related to the user during the sleep session. The one or more sensors 130 are configured to generated physiological data related to the user during the sleep session, as well as non-physiological data (such as data related to the operation of the respiration system). Often however, only basic data related to the user's use of the respiratory therapy system 120 (such as flow data or pressure data) is initially provided to the control system 110 and/or the memory device 114 and used to determine parameters or metrics related to the user. Additional data related to (i) the user's use of the respiratory therapy system 120, (ii) the respiratory therapy system 120 itself, (iii) aspects or characteristics of the user separate from the respiratory therapy system 120, or (iv) other general data, can be useful in determining more accurate values of the parameters, or determining the values of new parameters. Any additional data cannot be obtained and utilized without consent from the user of the respiratory therapy system however. In order to obtain consent from the user to receive and analyze additional data beyond the initial data provided to the control system 110 and/or the memory device 114, a variety of methods or techniques can be utilized. Generally, one or more steps of any of the following methods or techniques can be implemented using any element or aspects of the system 100 (FIGS. 1-2) described herein.

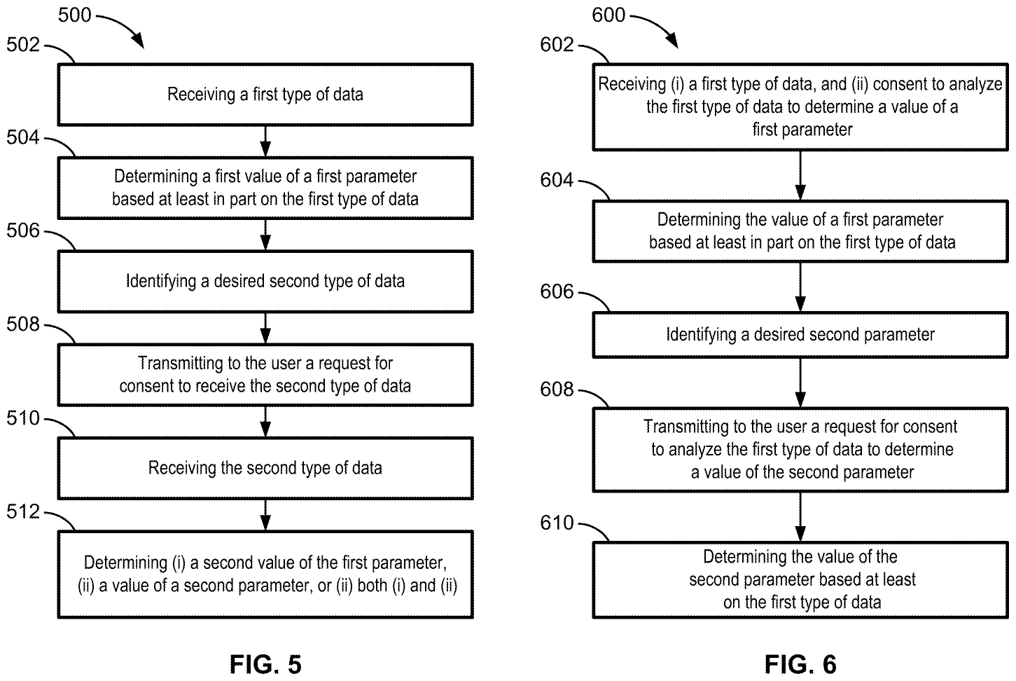

Referring now to FIG. 5, a method 500 for analyzing data related to the use of a respiratory therapy system (such as respiratory therapy system 120) by a user (such as user 210) during a sleep session is illustrated. The respiratory therapy system can include a respiratory therapy device (such as respiratory therapy device 122), a user interface (such as user interface 124), and a conduit (such as conduit 126). Generally, method 500 can be implemented using a system (such as system 100) that includes a control system (such as control system 110). The control system or a portion of the control system (such as the one or more processors 112) can be configured to carry out the various steps of method 500. A memory device (such as memory device 114) can be used to store any type of data utilized in the steps of method 500 (or other methods disclosed herein).

Step 502 of the method 500 includes receiving a first type of data related to the user during the sleep session. Generally, the first type of data can include any type of data related to the user's use of the respiratory therapy system. In some implementations, the first type of data is physiological data associated with the user during the sleep session. For example, the first type of data can be flow data and/or pressure data related to the user's respiration.

Step 504 of the method 500 includes determining a first value of a first parameter related to the user. The first value of the first parameter is based at least in part on the first type of data. In some implementations, the first parameter is a sleep-related parameter for the user during the sleep session that can be determined by analyzing physiological data. For example, if the first type of data is flow data and/or pressure data (e.g., respiration data related to the respiration of the user), a respiration rate of the user during the sleep session can be determined. Generally, the first parameter can be any sleep-related parameter, any physiological parameter, or other parameters.

Step 506 of the method 500 includes identifying a desired second type of data. Generally, the second type of data can be any data that the user has not already given consent to obtain and/or analyze. For example, the second type of data can include physiological data (such as addition respiration data), non-physiological data related to the user, non-physiological data related to the respiration system, etc. The second type of data can be related to the user's use of the respiratory therapy system. The second type of data can also be related to activities, events, information, etc. that are unrelated to the user's sleep session or use of the respiratory therapy system, or that occur outside of the sleep session and the use of the respiratory therapy system.

Step 508 of the method 500 includes transmitting to the user a request for consent to receive the second type of data. Because the user has not given consent to obtain and/or analyze the second type of data, the control system transmits the request for consent to the user. Step 510 of method 500 includes receiving the second type of data in response to receiving consent from the user to receive the second type of data. users can respond to the requests for consent in a variety of different manners, such as via a voice command (e.g., speaking to a smart speaker or smart device), via a biometric indicator (e.g., a fingerprint or a face scan), via a gesture in front of some type of sensor, via a physical input mechanism (e.g., pressing a touch screen, activating a button, typing on a keyboard, clicking a button on a mouse), or via any combination of these manners of input or others. Other types of audio or speech could also be used to provide consent. The request for consent can be responded to by using any of the components of the system, which could include a user device (such as the user device 170) a microphone (such as the microphone 140) or any of one or more sensors (such as sensors 130). In some implementations, the consent referred to herein can be sought from a third party, such as a family member, physician, healthcare provider, etc. This third party consent can be sought in circumstances such as when the user is physically or mentally incapacitated, and unable to respond to the request and/or provide the consent.

In some implementations, the control system may activate certain sensors to begin receiving the second type of data. In other implementations, the control system may begin to receive the second type of data from another source, for example a wired or wireless connection to the Internet. In still other implementations, the user or another person or system may actively send the second type of data to the control system.

Finally, step 512 of the method 500 includes determining a second value of the first parameter, a value of a second parameter, or both. Generally, either determination takes into account the second type of data. Thus, the second type of data is used to determine an additional value of the previously-determined parameter, or the value of an entirely new parameter. In many of these implementations, the second value of the first parameter is more accurate than the first value of the first parameter, and thus the second type of data is used to more accurately determine the value of the first parameter. In some implementations, the first value of the first parameter is determined with a first confidence interval or probability level. For example, the control system can determine the first value of the first parameter plus or minus X %, and the second value of the first parameter plus or minus Y %, where Y is less than X (e.g., the range of possible values of the second value of the first parameter is smaller than the first value of the first parameter). In another example, the control system can determine the first value of the first parameter with an X % confidence interval, and determine the second value of the first parameter with a Z % confidence level, where Z is greater than X

The second and more accurate value of the first parameter can be newly determined, or can be based on a modification of the first value of the first parameter. Thus, the second value of the first parameter can be based on any combination of the first type of data, the first value of the first parameter, and the second type of data. Similarly, the first value of the second parameter (e.g., the new parameter) can be based on any combination of the first type of data, the first value of the first parameter, and the second type of data.

In some implementations, the first type of data is received during or subsequent to a first sleep session, and the second type of data is received during or subsequent to a second sleep session. Thus, in one example, the value of the first parameter is determined while the user is asleep during the first sleep session, and then the next day when the user is awake, the user can consent to the control system receiving the second type of data once the user falls asleep that night during the second sleep session. This example can be utilized when the second type of data is generated during the sleep session. The request for consent to receive the second type of data can be transmitted during the sleep session (e.g., while the user is still asleep), or after the sleep session (e.g., once the user has awakened and got out of bed). The second type of data can also be received generally subsequent to the first sleep session, which includes both when the user is awake after the first sleep session, and once the second sleep session has commenced. In still other implementations, both the first and second types of data can be received during the first sleep session. For example, the user may affirmatively respond to the request for consent to receive the second type of data when the user is lying in bed during the first sleep session, but prior to falling asleep.

The identification of the desired second type of data in step 506 of method 500 can be based on a variety of different factors. In some implementations, the desired second type of data is based on the determined value of the first parameter. For example, the value of the first parameter may reveal that the user potentially has a certain medical condition or affliction, and thus the control system identifies a second type of data that may provide more insight as to whether the user has the medical condition or affliction. In other implementations, the identification of the second type of data may be based solely on what the first type of data is. For example, the control system may identify additional data that can be useful when analyzed in conjunction with the first type of data already available to the control system. In further implementations, the identification of the second type of data is based on how accurate the value of the first parameter is when determined from only the first type of data. If the accuracy does not satisfy some threshold accuracy, the control system can identify the second type of data as data that can be used to obtain a more accurate value.

In some implementations, step 508 alternatively or additionally includes transmitting a request for consent to analyze the second type of data. In some implementations, the control system may already have access to the second type of data. For example, the control system may have consent to store the second type of data in the memory device. However, the control system may not have consent from the user to analyze the second type of data. In these implementations, instead of transmitting a request for consent to receive the second type of data, the control system transmits to the user a request for consent to analyze the second type of data. In implementations where the control system and/or the memory device do not have access to the second type of data, the control system may transmit a request for consent to analyze the second type of data, in addition to transmitting the request for consent to receive the second type of data. These implementations can be used where separate consent is required to both receive and analyze the second type of data.

In some implementations, step 508 alternatively or additionally includes a request for consent to activate any of the one or more sensors in order to generate and receive the second type of data. In many implementations, one or more of the sensors are present during the sleep session (for example as part of the respiratory therapy system), but are not actively generating data. Thus, the control system can transmit a request for consent to activate a given sensor in order to generate and receive the second type of data. In one implementation, the first type of data is respiration data generated by a pressure sensor or a flow rate sensor, and in response to analyze the respiration data, a request to activate an acoustic sensor and receive audio data is transmitted to the user.

In some implementations, the method 500 further includes the step of transmitting to the user a request for consent to send the first and/or second type of data to a third party. The third party could be a healthcare provider (e.g., the user's doctor), a family member, a friend, a caretaker, etc. This request for consent can also be accompanied by an explanation as to why the first and/or second type of data should be sent to the third party. For example, the control system may provide an explanation that the user's healthcare provider can utilize the first and/or second type of data to better treat the user at a future appointment, or to better track a disease or condition that the user has.

In some implementations, the second type of data includes a portion of the user's medical history. For example, the second type of data could include past diseases or afflictions the user has experienced, or any ongoing medical problems not already known to the control system. The medical history could also include information about the user's family history, e.g., conditions, diseases, afflictions, problem suffered by any of the user's relatives. The medical history could be obtained directly from the user, or could be obtained from an external source separate from the user, such as the user's healthcare provider, the user's electronic medical record, or the Internet. The control system can then utilize the user's medical history to take a variety of different actions. For example, the control system, may be able to more accurately determine the value of the first parameter based on information obtained from the user's medical history.

In some implementations, the method 500 further includes the step of transmitting to the user a request for consent to analyze the second type of data to determine whether the user is asleep. Generally, the system monitors the user during the sleep session to determine the number of respiratory events per hour that the user experiences. However, the accuracy of the determination of the number of events per hour can be affected by whether or not the user is asleep. For example, the data analyzed by the control system may indicate that the user is experiencing a certain number of events even when the user is awake. By determining whether the user is asleep, the control system is able to more accurately determine when actual events occur.

In some implementations, the second type of data includes movement data indicative of movement of the user during the sleep session. The movement data may show that the user is frequently moving, which can indicate that the user has not yet fallen asleep. The movement data can also show that the user is not moving or is infrequently moving, which can indicate that the user is asleep. In other implementations, the second type of data includes movement data indicative of components of the respiratory therapy system, such as the user interface or the conduit. These components (or other components) may move during the sleep session when the user moves. Thus, any movement of these components (or other components) can be used to aid in determining whether the user is asleep. This movement data can also show vibration of various components of the respiratory therapy system, which can indicate that the respiratory therapy device is currently activate and causing air to flow, which may be used to determine whether the user is asleep.

In other implementations, the second type of data includes audio data indicative of noises generated by the user and/or the respiratory therapy system during use. The audio data can be generated by the microphone. For example, the audio data may reveal that the user is snoring, indicating that the user is asleep. The audio data may also reveal that the user is talking, which generally indicates that the user is awake. The audio data may also reveal that the respiratory therapy system is making noise, for example due to the operation of the motor in the respiratory therapy device, or due to the pressurized air flowing through the respiratory therapy system. The noise from the respiratory therapy system indicates that the respiratory therapy system is being used, which can aid in determining that the user is asleep.

In any of these implementations, the control system analyzes the second type of data to determine whether or not the user is asleep. Once that determination is made, the control system can more accurately determine the number of events per hour that the user experiences, as compared to the determination of the number of events per hour when it was unknown whether the user was asleep. In any of these implementations, when the control system transmits the request for consent to receive and analyze the second type of data to determine whether the user is asleep, the control system can also transmit to the user an explanation of the benefits of receiving and analyzing the second type of data.

The audio data can also be used to determine if any air is leaking from the mouth of the user. When the user interface is a nasal mask or a nasal pillow mask, air can leak from the user's mouth, particularly if the user tends to breathe through their mouth when sleeping without the respiratory therapy system. The leaking air is generally pressurized air from the respiratory therapy device, which is thus escaping from the user's mouth instead of being delivered to the user's airway. Thus, in some implementations, the control system can transmit a request for consent to receive and analyze audio data in order to determine if any air is leaking from the user's mouth. This could be based on the value of the first parameter. For example, the first type of data may be respiratory data, and the value of the first parameter may indicate some type of problem with the user's respiration. The control system can first check to see if air leaking from the user's mouth is causing this problem.

In some implementations, the type of respiratory therapy system that the user is using can affect the value of the first parameter. For example, the values of any determined sleep-related parameters may differ depending on the type of interface that the user is using, the type of conduit the user is using, etc. By determining various characteristics of the respiratory therapy system, more accurate parameters can be determined. Thus, in some implementations, the second type of data is analyzed to determine any desired characteristics of the respiratory therapy system, such as characteristics of the conduit or the interface. The control system can then determine a more accurate value of the first parameter based on both the first type of data and the characteristics of the components of the respiratory therapy system.

The health of the motor of the respiratory therapy device can also impact the value of the first parameter. Thus, in some implementations, the method 500 can include the step of transmitting to the user a request for consent to receive and analyze audio data to determine the health of the respiratory therapy system or of various components of the respiratory therapy system, such as the motor of the respiratory therapy device. Once the health of the motor is determined, the values of any parameters (such as the first parameter) can be more accurately determined. In one example, a respiratory therapy device with a malfunctioning motor may cause the control system to determine that the user is suffering from a much larger or smaller number of events per hour than is actually occurring. Thus, by determining the health of the motor, the control system can more accurately determine the number of events per hour that the user is experiencing. Other physiological and non-physiological parameters can also be more accurately determined in this manner.

In some implementations, the first parameter may be a parameter that is indicative of a quality of sleep of the user. For example, the first parameter could be a sleep score that takes into account, for example, the length of time the user has been asleep, the amount of time the user has spent in various stages of the sleep cycle (e.g., REM sleep, non-REM sleep), the number of events per hour the user has experienced, etc. In these implementations, the method 500 can further include the step of transmitting to the user a suggestion to improve the quality of the user's sleep, based at least on the value of the first parameter and the second type of data.

In some implementations, it may be desirable to analyze the first type of data to determine parameters other than the first parameter. Thus, the method 500 can further include the step of transmitting to the user a request for consent to analyze the first type of data to determine value of a parameter other than the first type of parameter. The request for consent can also include an explanation of any benefits in determining the additional parameter, which can incentivize the user to give consent. Once the control system receives the user's consent, the control system can analyze the first type of data (which it already has access to) to determine the value of the additional parameter. In some of these implementations, the first data is physiological data, and the additional parameter is a physiological parameter.

In certain implementations, method 500 also includes the step of transmitting to the user an explanation of a potential use for the second type of data. In certain situations, user's may be reluctant to provide more of their data to the control system. By explaining possible uses for the second type of data, the user is incentivized to respond affirmatively to the request for consent and to provide access to the second type of data. The explanation of the potential use for the second type of data, can include an indication that the second type of data enables the value of the first parameter to be determined more accurately than the first value (e.g., with a larger confidence internal or with a smaller range of possible values).

In some of these implementations, the value of the first parameter may be correlated with the user having a certain medical condition (e.g., heart rate data may indicate a possible heart condition). The explanation of the potential use for the second type of data can include an indication of the correlation between the value of the first parameter and the medical condition, to thereby incentivize the user to consent to the control system to receive the second type of data. In these implementations, the control system can estimate a percentage likelihood that the user has the medical condition, based on the second type of data. If this percentage likelihood satisfies a predetermined threshold, the control system can take number of actions, such as (i) transmitting a notification to the user or a third party, (ii) transmitting a suggested treatment routine (such as a suggestion of a medicine to take) to the user or a third party, or (iii) suggesting an appointment with a healthcare provider. The third party can include the healthcare provider, a friend of the user, a family member of the user, any other desired third party, or any combination of third parties.

In still other implementations, the second data may include some or all of the user's medical record (which can be an electronic medical record). The explanation of the potential use for the user's medical record can include an indication that access to the user's medical record can enable any desired additional parameters to be identified. For example, a certain value of the first parameter may not on its own indicate that any other parameters would be beneficial if known. However, if the control system has access to the user's medical record, the control system can determine if the user has any preexisting condition or disease that would result in additional parameters being beneficial, when viewed in combination with the value of the first parameter. For example, the first type of data may reveal certain respiratory or cardiac characteristics (e.g., respiration rate or variability, heart rate or variability) that on their own, do not indicate any type of health problem. However, if the control system accesses the user's medical record and determines that the user has a preexisting condition or disease, those same respiratory or cardiac characteristics may indicate a health problem that requires the determination of additional parameters. In some implementations, the control system determines the value of any additional parameters based on the first type of data after receiving consent from the user. In other implementations, after receiving consent from the user, the control system activates one of the sensors of the respiratory therapy system to receive additional data, and determines the value of the additional parameter based on this additional data.

As noted above, the first type of data or the second type of data include audio data generated by the microphone of the respiratory therapy system. The audio data can be associated with movement of the user during the sleep session, movement of one or more components of the respiratory therapy system (such as the user interface or the conduit) during the sleep session, air leaking from any component of the respiratory therapy system (e.g., the user interface), air leaking from the mouth of the user when the user interface is a nasal mask or a nasal pillow mask (indicating that a portion of the pressurized air being supplied to the user is escaping from the mouth of the user), or any combination thereof. The first type of data or the second type of data can also include movement data indicative of movement of the user during the sleep session, movement of a component of the respiratory therapy system during the sleep session, or both. The audio data and the movement data can be used in any manner in accordance with the various techniques disclosed herein.

In some implementations, the request for consent to receive the second type of data is based at least in part on the user's location. For example, different jurisdictions (e.g., different states, different countries) can have different laws and regulations regarding privacy and data collection. Thus, any requests for consent transmitted to the user may differ depending on the local laws and regulations. In these implementations, the control system is configured to determine a location of the user prior to transmitting the request for consent to receive the second type of data. And in some of these implementations, the control system requests consent from the user to determine the location of the user, prior to requesting consent to receive the second type of data. The location of the user can be determined to varying levels of specificity, for example by determining what continent the user is in, what country the user is in, what state the user is in, what province the user is in, what city or town the user is in, what neighborhood the user is in, etc. The control system may also determine a location of the user relative to some base location, for example by determining whether the user is at home or at a location different from their home. The control system can also determine the location of the user based on coordinates, e.g., latitude and longitude.

In some implementations, method 500 can be used to analyze the user's breathing to determine whether the user has a medical condition. In these implementations, the first data is respiration data associated with the user, and can be generated by a pressure sensor and/or a flow rate sensor (such as pressure sensor 132 and flow rate sensor 134). The control system transmits a request for consent to analyze the respiration data to determine a respiratory parameter or respiratory parameters of the user during the sleep session, and then analyzes the respiration data to determine the respiratory parameter. Based at least in part on the value of the respiratory parameter, the control system estimates a percentage likelihood that the user has a certain medical condition, and then transmits a notification to the user or a third party (such as a healthcare provider, a friend, a family member, etc.) indicating the percentage likelihood that the user has the medical condition. In some implementations, the respiratory parameter is an inspiration/expiration ratio, which can be indicative of whether the user has chronic obstructive pulmonary disease (COPD), bronchitis, emphysema, etc.

In some implementations, the control system can additionally or alternatively analyze audio data to detect respiratory problems with the user. For example, the control system can transmit a request for consent to analyze audio data generated by the microphone. If the user consents to the request, the control system can analyze the audio data to detect if the user is breathing irregularly, coughing, wheezing, choking, snoring, etc. during the sleep session, which can aid in estimating the percentage likelihood that the user has a certain medical condition or respiratory issue.

In certain implementations, the second type of data is personal data associated with the user, such as (i) an age of the user, (ii) a sex of the user, (iii) a gender of the user, (iv) a geographic location of the user, (v) a height of the user, (vi) a weight of the user, (vii) medical information associated with the user, (viii) a smoking status of the user, (ix) an occupation of the user, (x) an education level of the user, (xi) an income level of the user, (xii) a frequency and duration of any travel of the user, or (xiii) any combination of (i)-(xii). The medical information associated with the user can include any medical condition, disease, affliction, etc. that the user might be suffering from, such as hypertension, drug-resistant hypertension, diabetes, chronic obstructive pulmonary disease (COPD), asthma, obesity, depression, gastroesophageal reflux disease (GERD), hypercholesterolemia, diabetes mellitus, strokes, heart attacks, heart failure, or any combination thereof. The medical information can be analyzed to determine various comorbidities of the user.

In these implementations, the control system is configured to transmit to the user a request for consent to analyze the user's personal data in order to sort the user into one or more populations. The populations can include age-based populations (e.g., teenagers, ages 18-30, ages 31-50, ages 50-65, ages 65 and older), sex-based or gender-based populations, medical-based populations (e.g., smokers and non-smokers, normal weight and overweight), location-based populations (e.g. residents of a certain neighborhood, state, or country), or any other suitable populations that may be formed from the personal data. These are sample populations into which the user can be sorted. Generally, any suitable populations based on any of the personal data (or other data) can be used.

In some of these implementations, the first value of the first parameter can be modified based on any population which the user has been sorted, thereby determining the more accurate second value of the first parameter. For example, analysis of the first type of data may reveal a certain value of a parameter, but if the user is overweight and smokes, the control system may adjust the value of that parameter to more accurately find the true value of the parameter. Thus, the second value of the first parameter can be based at least in part on the first value of the first parameter, and on any populations into which the user is sorted.

In others of these implementations, additional desired parameters can be identified based at least in part on the values of the first parameter and the populations into which the user is sorted. For example, a specific value of a parameter (e.g., heart rate, inspiration/expiration ratio) may be considered normal for a 25-year old non-smoker with a normal body weight. However, if the user is older, smokes, and is overweight or obese, that same value of that same parameter may indicate a potential medical problem or condition. The control system can then identify any additional parameters to determine in order to better determine whether the user has the potential medical problem or condition.

The control system can also generate an alert based at least on the value of the first parameter and the populations into which the user is sorted. This alert can be stored and/or transmitted to the user or any desired third party, such as a healthcare provider, a friend, a family member, etc.

In some implementations, the user can withdraw previously-granted consent. In these implementations, the control system can actively stop receiving data for which the user has withdrawn consent for the control system to receive. The control system can also actively stop analyzing data for which the user has withdrawn consent for the control system to analyze. Generally, the user can withdraw their consent using any suitable manner, such as via a voice command, via a biometric indicator, via a gesture in front of some type of sensor, via a physical input mechanism, or via any combination of these manners of input or others. In some implementations, the control system is configured to periodically transmit messages to the user indicating the user's ability to withdrawn previously-granted consent. Further, in response to the user indicating that they wish to withdraw consent, the control system in some implementations can provide information to the user as to what features would no longer be accessible without the data that the user wishes to withdraw consent for.

Referring now to FIG. 6, a method 600 for analyzing data related to the use of a respiratory therapy system (such as respiratory therapy system 120) by a user (such as user 210) during a sleep session is illustrated. The respiratory therapy system can include a respiratory therapy device (such as respiratory therapy device 122), a user interface (such as user interface 124), and a conduit (such as conduit 126). Generally, method 600 can be implemented using a system (such as system 100) that includes a control system (such as control system 110). The control system or a portion of the control system (such as the one or more processors 112) can be configured to carry out the various steps of method 500. A memory device (such as memory device 114) can be used to store any type of data utilized in the steps of method 600 (or other methods disclosed herein).

Step 602 of the method 600 is similar to step 502 of method 500, and includes receiving a first type of data related to the user during the sleep session, and consent to analyze the first type of data to determine a value of a first parameter related to the user. Generally, the first type of data can include any type of data related to the user's use of a respiratory therapy system, and can be physiological or non-physiological data. Step 604 of the method 600 is similar to step 504 of method 500, and includes determining the value of a first parameter based at least on the first type of data. The first parameter can be a sleep-related parameter, or can be other physiological or non-physiological parameters.

Step 606 of method 600 is similar to step 506 of method 500, and includes identifying a desired second parameter. In some implementations, the identification of the desired second parameter is based at least in part on the value of the first parameter. For example, a high or low respiration rate or heart rate may indicate other types of parameters to check, in order to determine whether the high or low respiration rate or heart rate is problematic. The identification of the desired second parameter can also be based at least in part on the identity of the first parameter, regardless of the value of the first parameter.

Step 608 of method 600 includes transmitting to the user a request for consent to analyze the first type of data to determine a value of the second parameter. In certain situations, the user may have already given consent for the control system to analyze the first type of data for a specific purpose, such as determining the value of the first parameter. However, this consent is often limited to just this purpose, and thus the control system needs specific consent to analyze the first type of data for any other purpose, such as determining the value of the second parameter. For example, the first type of data may be audio data related to operation of the motor of the respiratory therapy system, and the first parameter may be indicative of the health of the motor. If the motor is failing, the control system may wish to measure respiration-related parameters to ensure that the respiratory therapy system is still providing a sufficient amount of pressurized air to the user. Once the user consents, step 610 of method 600 includes determining the value of the second parameter based at least on the first type of data.

In some implementations, method 600 additionally includes the step of identifying a desired third parameter based at least in part on (i) the value of the first parameter, (ii) the value of the second parameter, or (iii) both (i) and (ii).

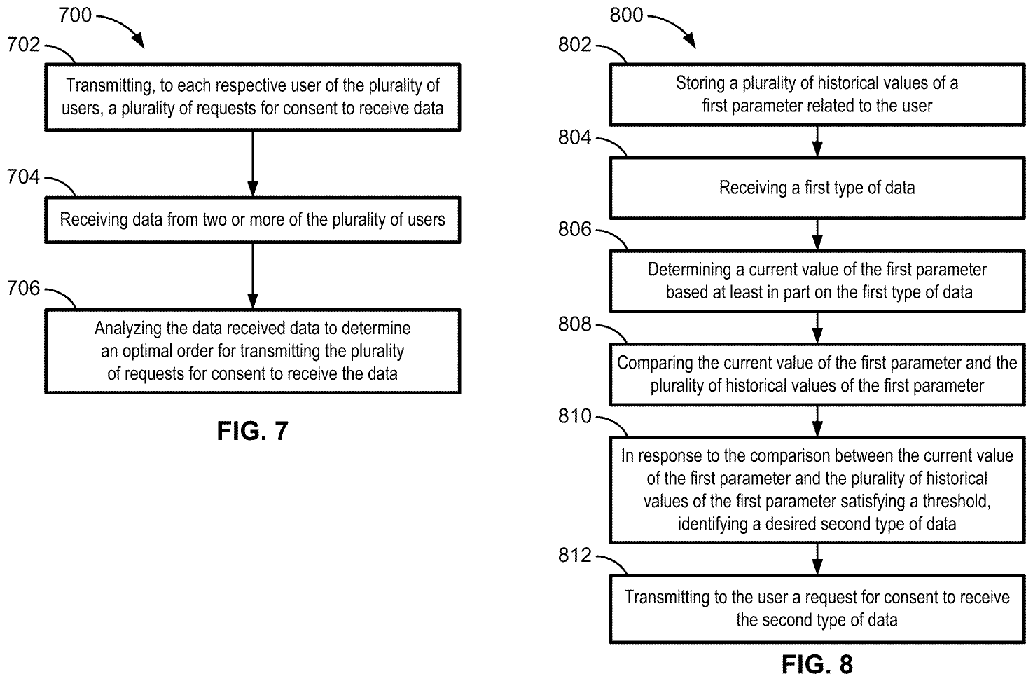

Referring now to FIG. 7, a method 700 for determining an optimal order for transmitting a plurality of requests for consent to receive and analyze data related to the use of a respiratory therapy system (such as respiratory therapy system 120) by one or more users (which can include user 210) is illustrated. The respiratory therapy system can include a respiratory therapy device (such as respiratory therapy device 122), a user interface (such as user interface 124), and a conduit (such as conduit 126). It may be desirable to determine an optimal order of transmitting requests to users for consent to receive a variety of different types of data. For example, user may be more likely to consent to sending in certain types of data if asked prior to requests for consent for other types of data. By comparing data collected from multiple different users, the optimal order can be determined. Generally, method 700 can be implemented using a system (such as system 100) that includes a control system (such as control system 110). The control system or a portion of the control system (such as the one or more processors 112) can be configured to carry out the various steps of method 500. A memory device (such as memory device 114) can be used to store any type of data utilized in the steps of method 700 (or other methods disclosed herein).

Step 702 of method 700 includes transmitting a plurality of requests for consent to receive data to a plurality of users. Generally, the data requested is associated with each user's use of their respiratory therapy system. However, some of the requests may be for other data as well. For each user, the plurality of requests is generally transmitted according to a respective order.

Step 704 of method 700 includes receiving data from two or more of the plurality of users, in response to receiving consent to do so from each user. In order to determine an optimal order, data must be collected from at least two users, in order to compare the data received. However, data can be collected from any number of users, so long as data from at least two users is collected.

Step 706 of method 700 includes analyzing all of the received data to determine an optimal order for transmitting the requests for consent to receive the data. The optimal order can be determined in a variety of different ways. In some implementations, the optimal order for transmitting the requests is the order that results in the most amount of data received from the user. Thus, order of requests for the user that consented to the sending in the largest amount of data can be used in the future with that user or other users, in order to collect the most amount of data. In other implementations, the optimal order is the order that results in a minimum amount of time between the beginning of the requests being transmitted to the user, and receiving some threshold of data. In certain situations, there may be an order that results in the most amount of received data. However, this order may require a much longer amount of time before the user consents to sending in that amount of data, making the use of that order impractical. Instead, some minimum amount of data can be identified, and the request order that obtains that amount of data (or more) in the minimum amount of time can be considered to be the optimal request order.

In some implementations, method 700 further includes the step of determining the optimal time of day for transmitting the plurality of requests. For example, users may be more receptive to consenting to requests for data in the afternoon or at night as compared to the morning. By analyzing all of the data received from the users, the optimal time of day can be determined.

In certain implementations, the received data can be personal data, such as but not limited includes (i) an age of the user, (ii) a sex of the user, (iii) a gender of the user, (iv) a geographic location of the user, (v) a height of the user, (vi) a weight of the user, (vii) medical information associated with the user, (viii) a smoking status of the user, (ix) an occupation of the user, (x) an education level of the user, (xi) an income level of the user, (xii) a frequency and duration of any travel of the user, or (xiii) any combination of (i)-(xii). The personal data can be analyzed to sort each user into one or more populations. The optimal order for each population of users can then be identified. For example, users in different age ranges may respond differently to the same order of requests for consent. The optimal order for transmitting the requests for consent for each age range can be determined, in order to collect more data.

The data can also be analyzed to determine the manner in which consent was received from the user, which can be used to aid in determining the optimal order for transmitting the requests for consent to receive data. As detailed herein, users can respond to the requests for consent in a variety of different manners, such as via a voice command (e.g., speaking to a smart speaker or smart device), via a biometric indicator (e.g., a fingerprint or a face scan), via a gesture in front of some type of sensor, via a physical input mechanism (e.g., pressing a touch screen, activating a button, typing on a keyboard, clicking a button on a mouse), or via any combination of these manners of input or others. users that utilize different ways to respond to the requests for consent may respond more optimally to receiving the requests for consent in different orders. Thus, the optimal order for transmitting the plurality of requests for consent to receive data can be based at least in part on the manner in which user responded to the requests for consent.

Referring now to FIG. 8, a method 800 for analyzing data related to use of a respiratory therapy system (such as respiratory therapy system 120) by a user (such as user 210) to determine a change in a parameter related to the user during a sleep session is illustrated. The respiratory therapy system can include a respiratory therapy device (such as respiratory therapy device 122), a user interface (such as user interface 124), and a conduit (such as conduit 126). Generally, method 800 can be implemented using a system (such as system 100) that includes a control system (such as control system 110). The control system or a portion of the control system (such as the one or more processors 112) can be configured to carry out the various steps of method 500. A memory device (such as memory device 114) can be used to store any type of data utilized in the steps of method 600 (or other methods disclosed herein).

Step 802 of method 800 includes storing a plurality of historical values of a first parameter. The historical values could be previous values of the first parameter from the current sleep session, previous values of the first parameter from one or more previous sleep sessions, or both. Step 804 of method 800 includes receiving a first type of data related to the user during the sleep session. Step 806 of method 800 includes determining a current value of the first parameter based at least in part on the received first type of data.

Step 808 of method 800 includes comparing the plurality of historical values of the first parameter and the current value of the first parameter. This comparison can be done in any number of ways. In some implementations, a statistical parameter based on the historical values is determined, and the statistical parameter is then compared to the current value. The statistical parameter could be, for example, an average of the plurality of historical values of the first parameter, a median of the plurality of historical values of the first parameter, a running average of the plurality of historical values of the first parameter, a running median of the plurality of historical values of the first parameter, or any other suitable statistical parameter. The statistical parameter can then be compared to the current value of the first parameter.

In other implementations, the comparison includes performing a statistical operation on the current value of the first parameter and the historical values of the first parameter. The statistical operation can include a change-point analysis, a t-test, a morphological comparison or analysis, or any other suitable statistical operation. Generally, the change-point analysis attempts to identify times when the probability distribution of the values of the first parameter (historical and current) changes. The t-test attempts to determine if the current value of the first parameter different significantly from a mean of the plurality of historical values.

At step 810 of method 800, if the comparison between the historical values of the first parameter and the current value of the first parameter satisfy some predetermined threshold (e.g., if the current value of the first parameter is too large, too small, indicates a potential medical problem, indicates a potential problem with the respiratory therapy system, etc.), a desired second type of data is identified. The second type of data can be any type of data that can aid in explaining why the current value of the first parameter satisfied the threshold. At step 812 of method 800, the control system can transmit o the user a request for consent to receive the second type of data. In some implementations, step 812 also includes transmitting an explanation of a potential use for the second type of data and/or transmitting a request for consent to analyze the second type of data to determine why the comparison between the current and historical values of the first parameter satisfied the threshold.

Thus, method 800 can be used to monitor a user in real-time during a sleep session, in order to determine if any parameters (such as sleep-related parameters or other physiological and non-physiological parameters) deviate from a normal or expected value or range of values during the sleep session. For example, method 800 can be used to attempt to determine why a user's heart rate or respiration rate spikes or dives during a sleep session. In another example, if the user's heart rate variability suddenly changes from an expected range, the control system can identify this problem and attempt to determine why. Method 800 and also further include transmitting a notification of any discovered information to the user or any desired third party, such as a healthcare provider, a family member, or a friend.

In some implementations, the various methods discussed herein can be used as part of a “cascading consent” feature, wherein analysis of one type of data continually leads to request consent to receive an analyze a different type of data. For example, the control system may analyze respiration data related to the user's respiration data during the sleep session to determine a parameter. Based on this analysis, the control system can request consent to receive and analyze audio data to determine characteristics of the respiratory therapy system (such as the type of user interface or type of conduit). The parameter related to the user's respiration can be modified, and the control system can then request consent to analyze the audio data to determine a health of the motor of the respiratory therapy device, which can also be used to modify the determined parameter. In other implementations, the control system may request consent to monitor flow rate data or pressure data to determine a heart rate or respiration rate of the user, request consent to share the data and the determined heart rate or respiration rate with a third party, and then request for consent to analyze the flow rate data or pressure data (or receive new data) to determine whether the user interface is fitting on the user's face properly. Generally, the control system can continually request consent to receive and analyze a variety of different types of data based on the data it currently has access to, in order to provide comprehensive care to the user.

One or more elements or aspects or steps, or any portion(s) thereof, from one or more of any of claims 1-93 below can be combined with one or more elements or aspects or steps, or any portion(s) thereof, from one or more of any of the other claims 1-93 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method of analyzing data related to use of a respiratory therapy system by a user during a sleep session, comprising:
receiving a first type of data related to the use of the respiratory therapy system by the user during the sleep session;
determining a first value of a first parameter related to the use of the respiratory therapy system by the user based at least in part on the first type of data;
identifying a desired second type of data;
transmitting to the user a request for consent to receive the second type of data;
in response to receiving consent from the user, receiving the second type of data; and
determining, based at least in part on the second type of data, (i) a second value of the first parameter, (ii) a value of a second parameter, or (iii) both (i) and (ii).

2. The method of claim 1, wherein the desired second type of data is based at least in part on the determined first value of the first parameter, the first type of data, an accuracy of the determined first value of the first parameter, or any combination thereof.

3-5. (canceled)

6. The method of claim 1, wherein the first type of data is physiological data associated with the user during the sleep session, and wherein the first parameter is a sleep-related parameter for the user during the sleep session.

7. The method of claim 6, wherein the second type of data is personal data associated with the user, and wherein the method further comprises:
transmitting to the user a request for consent to analyze the personal data to sort the user into one or more populations of users; and
determining a modified value of the first parameter based at least in part on the one or more populations of users into which the user is sorted, the modified value of the first parameter being more accurate than the first value of the first parameter.

8-12. (canceled)

13. The method of claim 7, further comprising generating, based at least in part on (i) the value of first parameter, (ii) the one or more population of users into which the user is sorted, or (iii) both (i) and (ii), an alert related to the user.

14. (canceled)

15. The method of claim 1, wherein the second value of the first parameter is more accurate than the first value of the first parameter, and wherein the second value is based at least in part on (i) the first type of data, (ii) the first value of the first parameter, or (iii) both (i) and (ii).

16-20. (canceled)

21. The method of claim 1, further comprising transmitting to the user an explanation of a potential use for the second type of data.

22. The method of claim 21, wherein the explanation of the potential use for the second type of data includes an indication that the second type of data enables the second value of the first parameter to be determined with a larger confidence interval than the first value of the first parameter.

23. (canceled)

24. The method of claim 21, wherein the explanation of the potential use for the second type of data includes an indication of a correlation between the first value of first parameter and a potential medical condition, and wherein the method further comprises estimating, based at least in part on the second type of data, a percentage likelihood that the user has the medical condition.

25. (canceled)

26. The method of claim 24, wherein the method further comprises, in response to the estimated percentage likelihood satisfying a threshold, transmitting a notification associated with the medical condition, a suggested treatment routine, a suggested appointment with a healthcare provider, or any combination thereof.

27-28. (canceled)

29. The method of claim 21, wherein the second data includes an electronic medical record of the user, and wherein the explanation of the potential use for the electronic medical record of the user includes an indication that the electronic medical record of the user enables a desired additional parameter based on the first data to be identified.

30-31. (canceled)

32. The method of claim 1, further comprising:
generating, based on the first data, a first estimation of a number of respiratory events per hour experienced by the user;
transmitting to the user a request for consent to analyze the second type of data to determine whether user is asleep,
generating, based on the first data and the determination of whether user is asleep, a second estimation of the number of respiratory events per hour experienced by the user, the second estimation being more accurate than the first estimation.

33-34. (canceled)

35. The method of claim 32, wherein the second type of data includes (i) movement data indicative of movement of the user during the sleep session, (ii) movement data indicative of movement of a component of the respiratory therapy system during the sleep session, (iii) audio data indicative of noise generated by the user during the sleep session, (iv) audio data indicative of noise generated by the respiratory therapy system during the sleep session, or (v) any combination of (i)-(iv).

36-37. (canceled)

38. The method of claim 1, wherein the second type of data is indicative of one or more characteristics of the respiratory therapy system, and wherein the second value of the first parameter is based at least in part on the one or more characteristics of the respiratory therapy system.

39. The method of claim 38, wherein the respiratory therapy system includes a respiratory therapy device, a conduit, and an interface, the user being connected to the respiratory therapy device via the conduit and the interface, and wherein one or more characteristics of the respiratory therapy system include (i) one or more characteristics of a conduit, (ii) one or more characteristics of the interface, or (iii) both (i) and (ii), and wherein the second value of the first parameter is more accurate than the first value of the first parameter.

40-49. (canceled)

50. The method of claim 1, wherein the first data is respiration data associated with the user of the respiratory therapy system by the user during the sleep session, and wherein the method further comprises:
transmitting, to the user, a request for consent to analyze the respiration data to determine an inspiration/expiration ratio of the user during the sleep session;
in response to receiving consent from the user, analyzing the respiration to determine the inspiration/expiration ratio of the user during the sleep session;
estimating a percentage likelihood that the user has a medical condition based at least in part on the determined inspiration/expiration ratio of the user; and
transmitting a notification to (i) the user, (ii) a healthcare provider, or (iii) both (i) and (ii), the notification indicating the percentage likelihood that the user has the medical condition.

51. (canceled)

52. The method of claim 50, wherein the respiratory therapy system includes a microphone, and wherein the method further comprises:
transmitting, to the user, a request for consent to receive audio data from the microphone;
in response to receiving consent from the user, receiving the audio data from the microphone; and
analyzing the audio data to detect coughing or wheezing from the user, the detected coughing or wheezing aiding in estimating the percentage likelihood that the user has the medical condition.

53. The method of claim 1, wherein the respiratory therapy system includes a first sensor configured to generate the first type of data, and a second sensor configured to generate the second type of data, and wherein the method further comprises transmitting to the user a request for consent to activate the second sensor and to generate the second type of data.

54-56. (canceled)

57. The method of claim 1, wherein the second type of data is related to (i) use of the respiratory therapy system by the user, (ii) activities of the user occurring outside of the sleep session, or (iii) both (i) and (ii).

58-68. (canceled)

69. A method of analyzing data associated with use of a plurality of respiratory therapy systems by a plurality of users, the method comprising:
transmitting, to each respective user of the plurality of users, a plurality of requests for consent to receive data associated with a use of a respective one of the plurality of respiratory therapy systems by the respective user, the plurality of requests being transmitted to each respective user according to a respective order;
in response to receiving consent, receiving data from two or more of the plurality of users; and
analyzing the data received from each respective user of the two or more of the plurality of users to determine an optimal order for transmitting the plurality of requests for consent to receive the data.

70. The method of claim 69, wherein the optimal order for transmitting the plurality of requests is the respective order resulting in (i) a maximum amount of requested data received, as compared to the other respective orders, or (ii) a minimum amount of time between transmitting the plurality of requests and receiving at least a portion of the requested data, as compared to the other respective orders for transmitting the plurality of requests.

71. (canceled)

72. The method of claim 69, wherein the method further comprises analyzing the data received from each respective user of the two or more of the plurality of users to determine an optimal time of day for transmitting the plurality of requests based at least in part on the analyzed data.

73. The method of claim 69, wherein the requested data includes personal data, and wherein the method further comprises:
sorting each of the plurality of users into one or more populations of users based at least in part on the personal data received from each of the plurality of users; and
determining the optimal order for transmitting the plurality of requests for the plurality of types of data for each of the one or more populations of users.

74. The method of claim 69, further comprising determining a manner in which consent was received from each of the two or more of the plurality of users, the optimal order for transmitting the plurality of requests being based at least in part on the determined manner in which consent was received from each of the two or more plurality of users.

75-77. (canceled)

78. A method of analyzing data related to use of a respiratory therapy system by a user during a current sleep session, comprising:
storing a plurality of historical values of a first parameter related to the user;
receiving a first type of data related to the user during the current sleep session;
determining a current value of the first parameter based at least in part on the first type of data;
comparing the current value of the first parameter and the plurality of historical values of the first parameter;
in response to the comparison between the current value of the first parameter and the plurality of historical values of the first parameter satisfying a threshold, identifying a desired second type of data; and
transmitting to the user a request for consent to receive the second type of data.

79. The method of claim 78, further comprising transmitting to the user an explanation of a potential use for the second type of data.

80. The method of claim 78, wherein the request for consent to receive the second type of data includes a request for consent to analyze the second type of data to determine a reason for the comparison between the current value of the first parameter and the plurality of historical values of the first parameter satisfying the threshold.

81. The method of claim 78, further comprising determining a statistical parameter based on the plurality of historical values of the first parameter, the statistical parameter being an average of the plurality of historical values of the first parameter, a median of the plurality of historical values of the first parameter, a running average of the plurality of historical values of the first parameter, or a running median of the plurality of historical values of the first parameter.

82. (canceled)

83. The method of claim 81, wherein the current value of the first parameter is compared to the (i) the historical values of the first parameter or (ii) the statistical parameter based on the plurality of historical values of the first parameter.

84. The method of claim 83, wherein the comparison includes performing a statistical operation on the current value of the first parameter and the statistical parameter based on the plurality of historical values of the first parameter.

85-93. (canceled)
